(19) United States
(12) Patent Application Publication
(10) Pub. No.: US 2010/0143438 A1
(43) Pub. Date: Jun. 10, 2010

(54) BIOMOLECULES

(75) Inventors: John Simon Todd, York (GB); Vincent Rein Ulijn, York (GB); Julie Gough, York (GB)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Strathclyde, Glasgow (GB)

(21) Appl. No.: 12/515,514

(22) PCT Filed: Nov. 19, 2007
(86) PCT No.: PCT/GB07/04417

§ 371 Date: Jan. 20, 2010

(30) Foreign Application Priority Data
Nov. 20, 2006 (GB) 0623160.9

(51) Int. Cl. A61F 2/00; C12M 1/22; C12M 1/24; C12N 11/02; A61K 38/06; C08B 15/06; C07K 14/78; C08G 63/91; C08G 18/83; C08F 20/18; C08F 112/08; C08F 283/02; C08G 79/02

(52) U.S. Cl. 424/423; 435/305.1; 435/304.1; 435/177; 514/18; 536/30; 530/356; 525/50; 525/410; 525/419; 525/453; 525/330.3; 525/333.3; 525/462; 525/538

(57) ABSTRACT

A substrate is provided having a biomolecule immobilised thereon, wherein the biomolecule is connected via an enzyme-cleavable link to a blocking moiety such that cleavage of the link causes removal of the blocking moiety and activation of the biomolecule.

### CROSS REFERENCE TO RELATED APPLICATION

This application claims priority to provisional application no. GB0623160.9 filed on 20 Nov. 2006, which is herein incorporated by reference.

### FIELD OF THE INVENTION

This invention relates to enzyme triggered activation of immobilised biomolecules thereby enabling selective activation of the biomolecule.

### BACKGROUND TO THE INVENTION

Dynamic cell-contacting surfaces are an increasingly important concept in the design of biomaterials. Such surfaces are capable of changing properties in response to applied stimuli thereby mimicking the dynamic properties of the materials that surround the cells in vivo, with the ultimate aim of controlling and directing cell behaviour. In this approach molecular-level changes in surface tethered biomolecules translate into macroscopic changes in the surface properties.

To date, these responsive surfaces have been developed to respond to stimuli such as temperature, ionic strength, solvent polarity, electric/magnetic field, light or the presence of small (bio-) molecules. Examples include surfaces that switch between (super-) hydrophobic and hydrophilic, or between bio-inert and bio-active to trigger capture or release of bio-macromolecules¹.

Such stimuli may be non-selective and disrupt biological interactions. For in vivo applications these stimuli are not feasible as, for example pH, ionic strength and solvent polarity are all more or less constant within the body. Stimuli such as light or magnetic/electric fields are not readily useable in vivo.

WO91/05036 discloses chemically derivatized surfaces to which small peptides, which comprise cell recognition sequences, have been covalently linked, the surfaces thereby having desirable cell adhesion effects. These surfaces do not, however, enable controlled or directed cell adhesion, any cell expressing the appropriate receptor for the cell recognition sequence will be capable of binding to the surface. This may be advantageous in tissue culture applications, where promoting the adhesion of a homogenous population of cells to a surface is desired. However, within in vitro or in vivo situations in which there is a heterogenous population of cells, it is often desirable to be able to control which cell type(s) adhere to the surface. For example, where it may be desirable to promote the adhesion of osteoblasts to the surface of an orthopaedic implant, whilst it would be undesirable to promote the adhesion of inflammatory cells to this surface.

In biological systems, dynamic processes are controlled by molecular feed-back systems involving on-demand enzyme triggered activation of biomolecules. We have surprisingly identified that it is possible to control and direct cell attachment to a surface based upon enzyme-triggered activation of surface tethered biomolecules under constant, physiological conditions.

The exploitation of enzyme catalysis as a trigger to change a materials' properties is particularly advantageous as it exploits the enzyme's (a) high selectively/specificity, (b) the ability to work under constant conditions of pH, temperature and ionic strength and (c) key involvement of biological pathways².

### SUMMARY OF THE INVENTION

According to an aspect of the invention there is provided a substrate having a biomolecule immobilised thereon, wherein the biomolecule is connected via an enzyme-cleavable link to a blocking moiety such that cleavage of the link causes removal of the blocking moiety.

In embodiments of the invention, the release of the blocking moiety causes activation of the biomolecule and/or the blocking moiety.

It is envisaged that the substrate is the surface of a device, or alternatively the substrate can be applied as a coating to at least part of a surface of a device.

Within an in vitro cell/tissue system, the substrate can be a surface of, for example, a cell/tissue culture flask; a cell/tissue culture plate, a cell/tissue culture dish, a Petri dish; a microcarrier or a macrocarrier. Alternatively, the substrate can be a coating applied to a surface of such devices.

Within an in vivo system, the substrate can be a surface of, for example, an implantable medical device, biomaterial or prosthesis. Alternatively, the substrate can be a coating applied to a surface of such a device. Implantable medical devices, biomaterials or prostheses include, artificial tissue implants (for example: orthopaedic implants, dental implants, soft tissue implants, cardiovascular implants), bioscaffolds, surgical fixation elements (for example: sutures, bone plates, bone screws, bone pins, bone nails), stents, nerve guides, nerve sheaths and wound dressings.

It is also envisaged that this technology can be applied to whole cell biosensors. Such systems utilise bacteria which are specifically engineered to react to the presence of chemical signals with the production of an easily quantifiable marker protein. In most cases, an existing regulatory system in the bacterial cell is exploited to drive expression of a specific reporter gene, such as bacterial luciferase, green fluorescent protein, beta-galactosidase or others. This is achieved by fusing the DNA for a promoterless reporter gene to an extra copy of the selected regulatable promoter and introducing this construction into the bacterial cell. Regulatory systems that have been applied include those for heavy metal resistancies (to obtain heavy metal responsive sensors), for organic compound degradation (to obtain organic compound sensors), and for cellular stress responses (to obtain general toxicity sensors).

In an embodiment of the invention, the bacteria is the substrate, the regulatable promoter is the biomolecule (being regulated via an enzymatic cleavage event caused by the target agent) and the blocking group once cleaved from the promoter is the marker.

In embodiments of the invention the substrate comprises a natural or synthetic polymer. Examples of suitable natural polymers include collagen, gelatin, hyaluronan, cellulose, chitin, dextran, fibrin, casein. Examples of suitable synthetic polymers include polylactide (PLA), polyglycolide (PGA), poly(lactide-co-glycolide) (PLGA), poly(e-caprolactone), polydioxanone, polyanhydride, poly(ethylene terephthalate), poly(urethane), poly(methylmethacrylate), poly(styrene), trimethylene carbonate, poly(β-hydroxybutyrate), poly(g-ethyl glutamate), poly(DTH iminocarbonate), poly(bisphenol A iminocarbonate), poly(ortho ester), polycyanoacrylate polyphosphazene, or poly(ethylene glycol)-acrylamide (PEGA).

In alternative embodiments of the invention, the substrate can be a ceramic or a metal or any other suitable natural or synthetic material for use in a medical device, biomaterial or prosthesis.

Covalent bonding is a form of chemical bonding that is characterized by the sharing of one or more electrons between two atoms. Non-covalent bonding refers to a variety of interactions that are non-covalent in nature, between molecules or parts of molecules that provide force to hold the molecules or parts of molecules together usually in a specific orientation or conformation. These non-covalent interactions include: ionic bonds, hydrophobic interactions, hydrogen bonds, Van der Waals forces and dipole-dipole bonds. The immobilisation of the biomolecule on the substrate can be via covalent or non-covalent bonding. In embodiments of the invention in which a plurality of biomolecules are immobilised to the surface, the biomolecules can be immobilised on the substrate via covalent or non-covalent bonding or a combination thereof.

In applications which take advantage of specific interactions between responsive surfaces, limiting non-specific interaction of cells, proteins and micro-organisms with the surface is critical, since such interactions can prove highly problematic for device efficacy and safety. A common method to reduce or prevent “bio-fouling” is the immobilisation of an antifouling polymer on a surface. Examples of antifouling polymers include hydrophilic polymers such as polyacrylates, oligosaccharides, polysaccharides, polymer mimics of phospholipids, phosphocholine, poly(ethylene) glycol (PEG), 3,4-dihydroxy-L-phenylalanine (DOPA)-PEG or polyethylene glycol acrylamide (PEGA) polymer³.

In a particularly advantageous embodiment of the invention the substrate is coated with a PEGA polymer. Such polymers are compatible both with organic solvent conditions and aqueous conditions required for biological assays⁴. When patterned onto surfaces, these hydrogels prevent non-specific cell adhesion, are suitable environments for enzyme catalysis, and are optically transparent, allowing for unimpaired assessment of results⁵.

The term biomolecule as referred to herein encompasses any compound that occurs naturally in living organisms. Biomolecules consist primarily of carbon and hydrogen, along with nitrogen, oxygen, phosphorous and sulphur. A diverse range of biomolecules exist and include lipids, phospholipids, glycolipids, sterols, vitamins, hormones, neurotransmitters, carbohydrates, monosaccharides, disaccharides, phosphates, amino acids, nucleic acids, nucleotides, peptides, oligopeptides, polypeptides and proteins and any other molecules that are capable of binding noncovalently to specific and complimentary portions of molecules or cells. Examples of such specific binding includes receptors binding to ligands, antigens binding to antibodies, and enzyme substrates binding to enzymes.

Biomolecules of the present invention are typically those that are intended to enhance or alter the function or performance of a device, in particular a medical device, biomaterial or prosthesis within a physiological environment. In embodiments of the invention, the biomolecule comprises cell attachment factors, growth factors, antithrombotic factors, binding receptors, ligands, enzymes, nucleic acids, antibodies, antigens and reporter molecules.

Cell attachment factors bind to specific cell surface receptors, thereby mechanically retaining the cell either to a substrate or another cell. In addition to promoting cell attachment, each type of attachment factor can promote other cell responses, including cell migration and differentiation⁶. Suitable cell attachment factors include the adhesion molecules laminin, fibronectin, collagen, vitronectin, tenascin, fibrinogen, thrombospondin, osteopontin, von Willebrand Factor and bone sialoprotein. In embodiments of the invention, the immobilised biomolecule comprises a peptide comprising an amino acid sequence or functional analogue thereof that possesses the biological activity of a specific domain or motif of a native cell attachment factor. It has been noted that surfaces on which long peptide chains have been immobilised are particularly unstable because the long oligopeptides are highly susceptible to degradation by high temperatures and to non-specific proteolytic action⁷. The peptide is therefore preferably less than 12 amino acids in length and comprises a cell attachment recognition domain or motif.

Examples of suitable domains or motifs within fibronectin include, but are not limited to, RGD⁸ (Arg Gly Asp) and REDV⁹ (Arg Glu Asp Val; SEQ ID NO:1).

RGD is a widely recognised cell recognition motif which is also found in laminin, entracin, thrombin, tenacin, fibrinogen, vitronectin, collagen and osteopondin.

Examples of suitable domains or motifs within laminin include, but are not limited to, YIGSR⁸ (Tyr Ile Gly Ser Arg) and SIKVAV⁸ (Ser Ile Lys Val-Ala-Val).

Examples of suitable domains or motifs within type IV collagen include GEFYFDLRLKGDK¹⁰ (Gly Glu Phe Tyr Phe Asp Leu Arg Leu Lys Gly Asp Lys).

Enzymatic cleavage of the link between the immobilised biomolecule and the blocking moiety by an enzyme released by the cell or added to the system, results in the exposure of the cell attachment factor and the consequent binding of the cell to the biomolecule. In this manner the controlled and directed binding of an appropriate cell population to an implantable device is achievable. For example, an enzyme released from a chondrocyte can cause the release of a blocking moiety from a biomolecule immobilised on the surface of an artificial cartilage implant, resulting, for example in the exposure of a chondrocyte cell recognition motif, and subsequent binding of the chondrocyte population to the implant. As further examples, the present invention can be used to direct a population of stem cells to the surface of a device.

In further embodiments of the inventions the immobilised biomolecule comprises a peptide having a cellular guidance function. For example, the peptide IKVAV (isoleucine-lysine-valine-alanine-valine) from laminin-1 promotes the growth of nerve endings and can be incorporated into a scaffold to promote nerve regeneration.

The implantation of a medical device, biomaterial or prosthesis elicits a host inflammatory response which in turn can influence the long-term behaviour of the implanted device. This host defense response against the “foreign body” may be the source of harm or destruction to the implant or may result in untoward inflammatory and healing responses which lead to failure of the device in its intended function. For example, osteolysis and aseptic loosening are known to cause failure of total hip replacements. As the femoral head articulates against the acetabular cup, wear debris are released which are of a clinically relevant size (0.1˜10 μm) and activate macrophages. Activated macrophages synthesize cytokines and growth factors that initiate inflammation and bone resorption, leading to failure of the implant.

A medical device, biomaterial or prosthesis designed to attenuate this inflammatory response is highly desirable.

In embodiments of the invention, the immobilised biomolecule comprises a binding receptor, such as an antibody or antigen. Antibodies present on the surface can bind to and remove specific antigens from the media that comes into contact with the immobilized antibodies. Similarly, antigens present on the surface can bind to and remove specific antibodies from the media that comes into contact with the immobilized antigens.

In further embodiments of the invention the immobilised biomolecule comprises a peptide comprising a sequence having an anti-inflammatory or immunomodulatory function. Research has shown that the immunomodulatory peptide α-melanocyte-stimulating hormone (α-MSH) and its carboxy-terminal tripeptide KPV (Lys-Pro-Val α-MSH)¹¹ have potent anti-inflammatory properties.

In allergic reactions an allergen interacts with and cross-links surface IgE antibodies on mast cells and basophils. Once the mast cell-antibody-antigen complex is formed, a complex series of events occurs that eventually leads to cell degranulation and the release of histamine (and other chemical mediators) from the mast cell or basophil. Once released, histamine can react with local or widespread tissues through histamine receptors to cause the following events: pruritus, vasodilatation, hypotension, flushing, headache, tachycardia, bronchoconstriction, increases is allergic response. An implanted device can be placed into an individual who is prone to recurrent allergic reactions. The immobilised biomolecule can be H₁-receptor antagonist, also known as a H₁-antihistamine. Cleavage of the enzyme-cleavable link by an enzyme released from the mast cells and/or basophils during the initial phases of the allergic reaction can result in exposure of the H₁-receptor antagonist, which can then bind H₁-receptors on circulating cells and attenuate the allergic reaction.

Infection can be a serious complication associated with the implantation of devices into the body. It can result in the failure of the implant and can be detrimental to the health of the patient. Whilst every effort is made during surgical procedures to maintain the sterility of an implant and implantation site, post-implantation infections do occur. The sustained delivery of antimicrobials and antibiotics is often not feasible and can result in tachyphylaxis. Responsive anti-infective surfaces whereby microbial, in particular bacterial secreted enzymes, such as aminopeptidases, cleave the enzyme link to activate an anti-microbial functionality of the immobilised biomolecule and/or released blocking moiety are desirable.

It is further envisaged that the present invention can be employed to monitor implant infection. For example, when the enzyme-cleavable link is designed to be selectively cleaved by a specific microbial agent, then the presence of a cleaved blocking moiety in a routine biological sample, such as a blood or urine sample, is indicative of the presence of that microbial agent at the implantation site.

It is envisaged that in embodiments of the invention a plurality of the same type of biomolecule are immobilised onto the substrate. For example, a plurality of the same peptide with the same function in the active form are immobilised.

It is further envisaged that a plurality of different types of biomolecule, having different functions, can be immobilised onto the substrate. For example, a first set of immobilised peptides each comprising a cell recognition motif which once activated enhance cell attachment to the substrate and a second set of immobilised peptides which once activated comprise anti-microbial properties. Each set of peptide can be activated by the same enzyme or different enzymes. For example when immobilised onto the surface of a hip implant the first set of peptides can be activated by an enzyme secreted from an osteoblast whilst the second set of peptides can be activated by an enzyme secreted from a bacterial cell, for example a Staphylococcus spp. cell. The different sets of peptides are therefore not necessarily activated at the same time or indeed activated at all, as activation is dependent on the specificity of the enzymes being secreted from the local cell population.

The enzyme cleavable link can be, for example, a peptide, ester, glycoside or oligonucleotide which can be located either between the blocking group and the biomolecule, within the biomolecule or within the blocking group. The enzyme cleavable link contains at least one enzyme recognition motif for an enzyme of the oxidoreductase, transferase, hydrolase, lyase, isomerase or ligase class of enzymes.

In embodiments of the invention, the hydrolase is a protease, also referred to as proteinase, peptidase or proteolytic enzyme. Proteases are classified based upon their catalytic mechanism into aspartic-, glutamic-, serine-, cysteine-, metallo- or threonine-protease.

In further embodiments of the invention, an amino acid having an aromatic side chain, for example, Phe (F), Tyr (Y) or Trp (W) is located at P1 of the enzyme cleavable link.

In an embodiment of the invention, the biomolecule is a peptide comprising Arg-Gly-Asp (RGD) connected to an enzyme cleavable link comprising Phe (F), such that an enzyme can selectively hydrolyse the Arg-Phe bond.

In an embodiment of the invention, the biomolecule is a peptide consisting of Arg-Gly-Asp (RGD) connected to an enzyme cleavable link consisting of Phe (F), such that an enzyme can selectively hydrolyse the Arg-Phe bond.

In an embodiment of the invention, Fmoc-F↓RGD-PEG is immobilised to the surface.

In a further embodiment of the invention, the biomolecule is a peptide comprising Arg-Gly-Asp (RGD) connected to an enzyme cleavable link comprising Ala (A)-Ala (A) such that an enzyme can selectively hydrolyse the Arg-Ala bond.

In an embodiment of the invention, the biomolecule is a peptide consisting of Ala (A)-Ala (A) such that an enzyme can selectively hydrolyse the Arg-Ala bond.

In an embodiment of the invention, Fmoc-A↓ARGD-PEG is immobilised to the surface.

Examples of suitable enzymes capable of specifically cleaving the Arg-Phe bond or Arg-Ala bond are serine proteases, for example, chymotrypsin, elastase or proteinase K and metalloproteases, for example, thermolysin.

In the present invention, a blocking moiety sterically or functionally inactivates the biomolecule until an appropriate enzyme cleaves the enzyme cleavable link. The specificity of this cleavage is determined by the enzyme recognition motif(s) located within the link. Cleavage activates the biomolecule.

It is particularly desirable that the blocking moiety is also bioactive following cleavage. A blocking moiety comprising N-fluorenylmethoxycarbonyl (Fmoc) is particularly advantageous as it sterically hinders the biomolecule and upon cleavage has its own inherent anti-inflammatory properties¹³. Thus, advantageously upon enzymatic cleavage of the link, the biomolecule demonstrates a first bioactive function and the blocking moiety demonstrates a second bioactive function.

Other suitable blocking groups include Boc, Troc, CBz, Mtt, Pmc, tBu, Tos, Mbzl and 2-Chloro-Z.

It is further envisaged that examples of the antifouling molecules mentioned above, such as oligosaccharides, polysaccharides, poly(ethylene) glycol (PEG) and phosphocholine can also function as a blocking group which sterically hinders the function of the biomolecule.

In an embodiment of the invention the biomolecule is a peptide comprising the cell recognition motif RGD, the blocking moiety is Fmoc, and the enzyme cleavable link comprises Phe (F) in the P1 position. Any enzyme having specificity for Phe (F) in this position can cleave the link, exposing the RGD motif which enhances cell attachment to the substrate, with the Fmoc moiety having anti-inflammatory properties being released.

According to a further aspect of the invention, there is provided a method of enhancing cell adhesion to a substrate comprising;

i) immobilising a biomolecule onto the substrate, the biomolecule comprising a cell recognition motif and being connected via an enzyme-cleavable link to a blocking moiety such that cleavage of the link causes removal of the blocking moiety and subsequent activation of the biomolecule; and

ii) exposing the biomolecule to an enzyme capable of cleaving the link.

The enzyme can be an exogenous or endogenous enzyme.

The method can be used for in vitro cell/tissue culture in which the substrate can be a surface of, for example, a cell/tissue culture flask; a cell/tissue culture plate, a cell/tissue culture dish, a Petri dish; a microcarrier or a macrocarrier. Alternatively the substrate can be a coating applied to a surface of such devices.

The method can be used for in vivo surgical procedures on an animal or human body in which the substrate can be a surface of, for example, an implantable medical device, biomaterial or prosthesis. Alternatively the substrate can be a coating applied to a surface of such a device. Implantable medical devices, biomaterials or prostheses include, artificial tissue implants (for example: orthopaedic implants, dental implants, soft tissue implants, cardiovascular implants), bioscaffolds, surgical fixation elements (for example: sutures, bone plates, bone screws, bone pins, bone nails), stents, nerve guides, nerve sheaths and wound dressings.

In an embodiment of the invention, the biomolecule is a peptide comprising the cell recognition motif RGD, the blocking moiety is Fmoc and the enzyme cleavable link comprises Phe (F) in the P1 position. Any enzyme having specificity for Phe (F) in this position can cleave the link, exposing the RGD motif which enhances cell attachment to the substrate, with the Fmoc moiety having anti-inflammatory properties being released.

According to a further aspect of the invention, there is provided a method of attenuating an inflammatory response in a subject following implantation of a medical device, the method comprising the step of immobilising a biomolecule onto a surface of the device, the biomolecule being connected via an enzyme cleavable link to a blocking moiety such that cleavage of the link causes removal of the blocking moiety and activation of the biomolecule and wherein the activated biomolecule is an anti-inflammatory agent.

In an embodiment of this aspect of the invention, the activated biomolecule comprises a peptide comprising lysine (K)-proline (P)-valine (V).

In a further embodiment of this aspect of the invention, the activated biomolecule comprises of a peptide consisting of lysine (K)-proline (P)-valine (V).

In a still further embodiment of the invention, the enzymatic cleavage further causes activation of the blocking moiety wherein the activated blocking moiety is an anti-inflammatory agent, such as Fmoc.

According to a further aspect of the invention, there is provided a diagnostic tool and/or biological assay.

In embodiments of the invention, the method could be used to qualitatively and/or quantitatively determine the presence of pathogens in a biological fluid, based on the premise that different pathogens have different enzyme release profiles.

In other embodiments of the invention, the method could be used to qualitatively and/or quantitatively determine the presence of a cell population in a biological fluid, based on the premise that different cell populations have different enzyme release profiles.

It is envisaged that a reporter molecule within the biomolecule could be exposed by the cleavage of the linker (by a pathogen-released enzyme) and subsequent release of the blocking moiety, with the reporter molecule being capable of detection in situ.

In further embodiments of the invention, a combination of enzymes can be used to switch “on” and “off” the adherence of cells to a surface. For example, on a Fmoc-FRGD-PEGA coated surface, chymotrypsin can be used to cleave the enzyme cleavable link, allowing cells to attach via the RGD motif. Subsequently, trypsin can be used to release the cells from the biomolecule.

Alternatively, the blocking moiety could act as the reporter molecule, with the detection of released blocking moiety confirming the presence of a particular pathogen.

### SPECIFIC EMBODIMENTS OF THE INVENTION

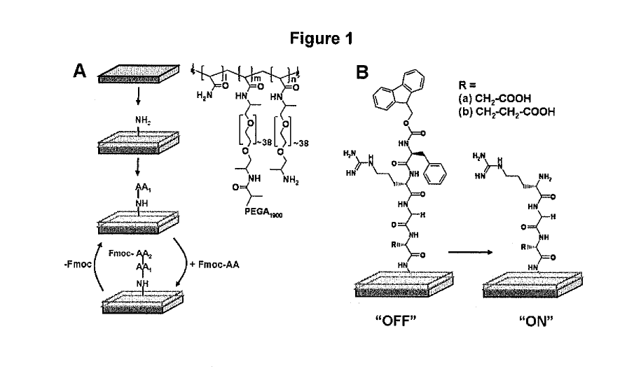

FIG. 1: a schematic of the preparation of peptide-functionalized PEGA surfaces capped with Fmoc-F.

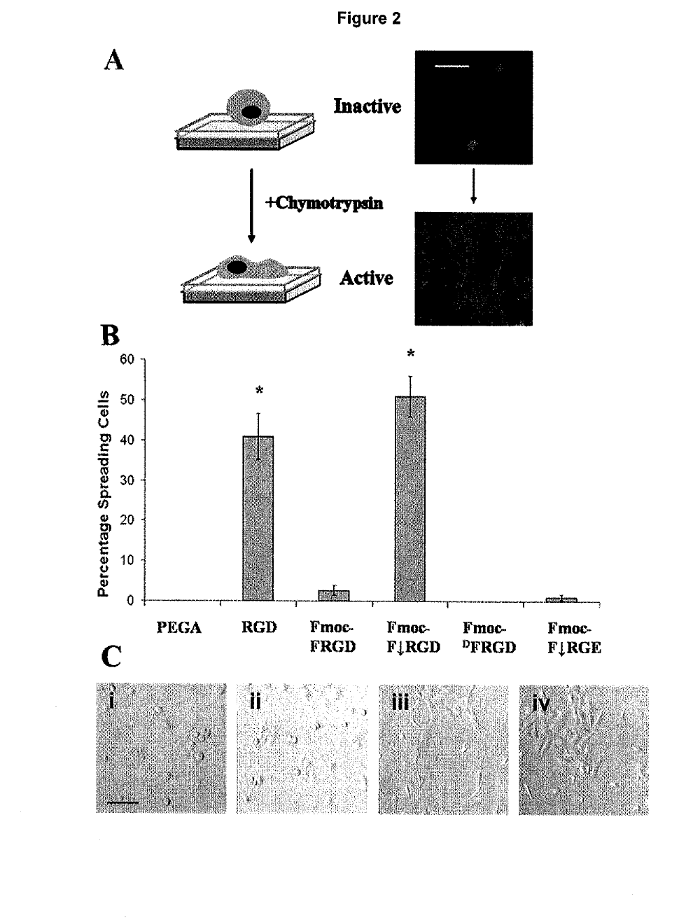

FIG. 2: a cellular response of primary derived human osteoblasts to modified PEGA surfaces.

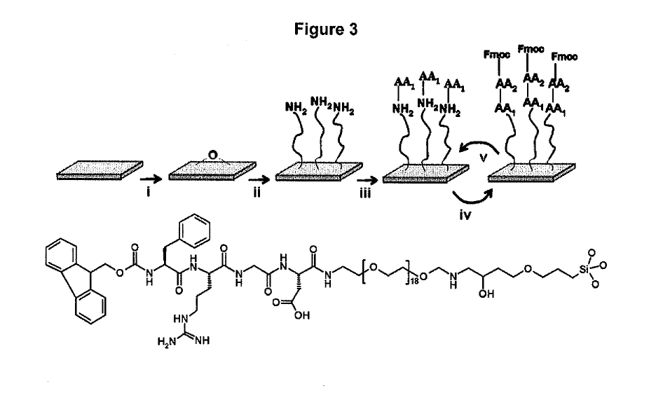

FIG. 3: a schematic of the preparation of a surface with a functionalised PEG monolayer.

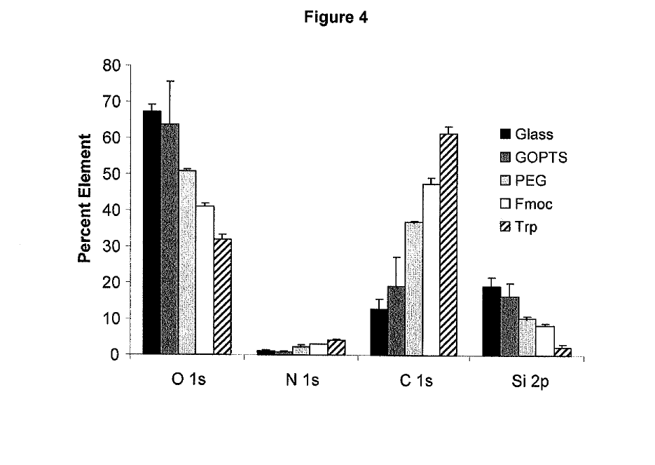

FIG. 4: a XPS Analysis of Single Amino Acid-PEG surfaces.

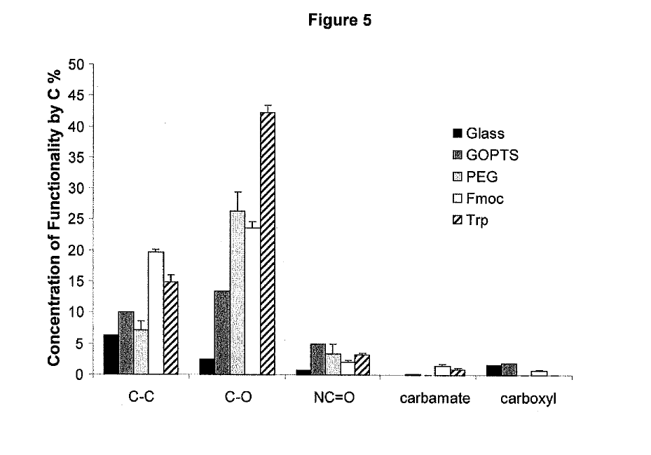

FIG. 5: a XPS Analysis of Single Amino Acid-PEG Surfaces modified with an Fmoc-amino acid (Fmoc-Trp).

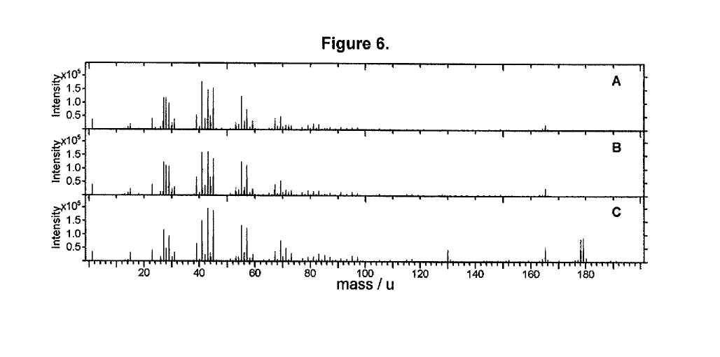

FIG. 6: ToF SIMS spectra of (A) Fmoc-F↓RGD-PEG, (B) Fmoc-FRGD-PEG and (C) Fmoc-Trp-PEG (comparison).

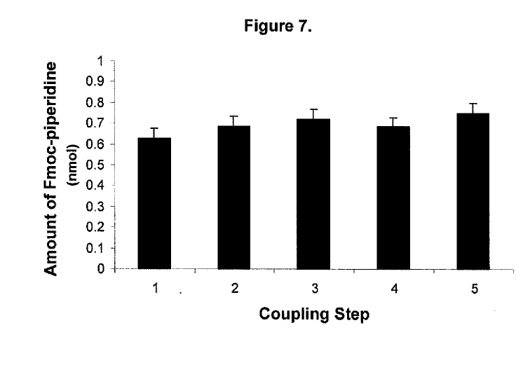

FIG. 7: Efficiency of Fmoc removal during stepwise synthesis of peptide on amino PEG surfaces.

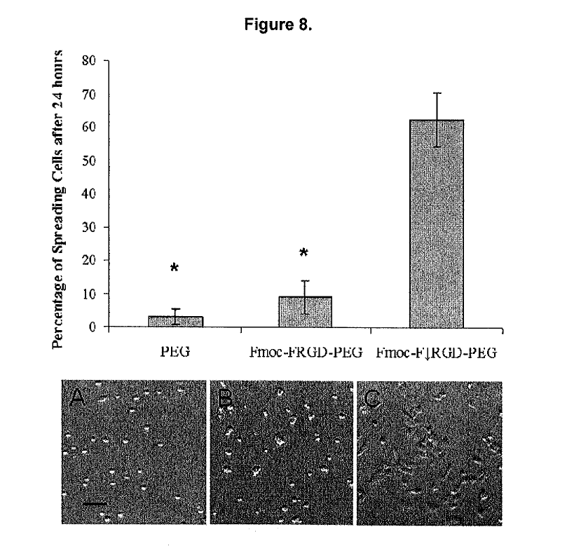

FIG. 8: Cellular responses of primary derived human osteoblasts to modified amino PEG surfaces.

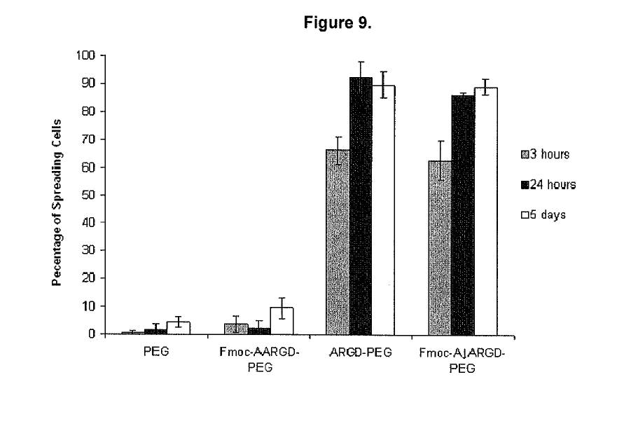

FIG. 9: Percentage of spreading osteoblast on different PEG surfaces after 3 and 24 hours and 5 days. Error bars represent standard deviations (n=15).

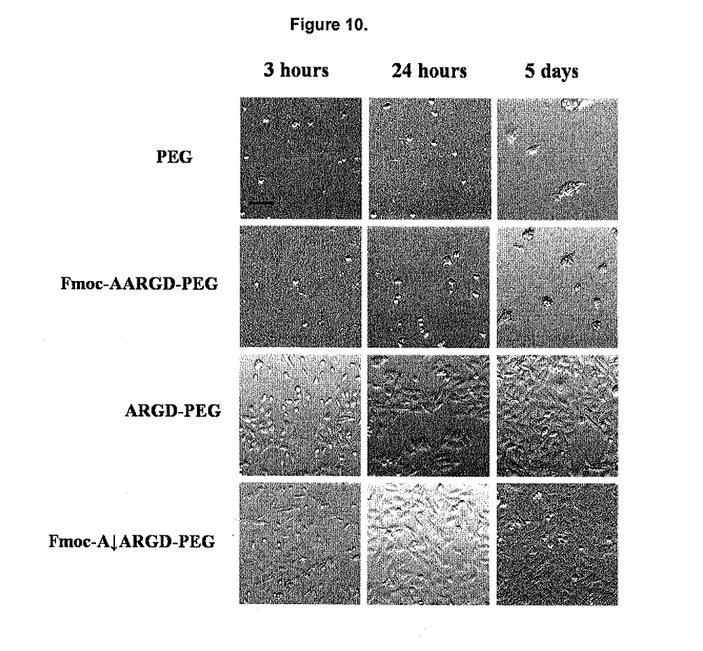

FIG. 10: Light micrographs of osteoblasts on various PEG surfaces after 3 hours, 24 hours and 5 days. The scale bar represents 50 microns.

### (A) ENZYME RESPONSE CELL ATTACHMENT TO HYDROGEL SURFACES

FIG. 1 illustrates an example of enzyme-triggered activation of a surface tethered bio-active molecule to dynamically control cell attachment. The method is based on a modification of the integrin binding peptide arginine-glycine-aspartic acid (RGD) to render it switchable between a non-cell adhesive (‘OFF’) state and an adhesive (‘ON’) state. The approach consists of chemically inactivating the cell-adhesive properties of surface tethered RGD sequences; by capping with a bulky blocking group (fluorenyl-9-methyoxycarbonyl-phenylalanine, Fmoc-F). The blocking group was chosen to contain an enzyme recognition motif (phenylalanine), so that the RGD sequence is activated biochemically, by an enzyme that can hydrolyze the Fmoc-F↓RGD peptide link. This method uniquely allows for triggered cell attachment under constant conditions of pH, temperature and ionic strength.

### Materials and Methods

### Production of PEGA Surfaces

PEGA hydrogel surfaces were prepared as shown in FIG. 1, A. The monomers (mono- and bis-acrylamido PEG (Mw=1900) (Versamatrix; Copenhagen, Denmark)) were mixed in a 1:1 ratio (w/w) with dimethylacrylamide and dissolved in dimethylacrylamide (DMF) and less than 1% Darocur 1173® (Ciba; Basel, Switzerland) (e.g., 0.5 g PEGA₁₉₀₀ monomers, 0.5 g dimethylacrylamide, 1.5 ml DMF, 0.02 g Darocur 1173®). The solution was stirred for at least 5 hours in the dark using a magnetic stirrer. To produce PEGA surfaces a few drops of PEGA solution were spin coated onto epoxy-functionalised slides (Genetix; Hampshire, UK) for 20 seconds at 1200 RPM. Protective polypropylene sheets were placed over the surface to prevent drying of the hydrogel and the surfaces exposed to UV light (365 nm) for approximately 45 seconds.

Modification of PEGA surfaces

Fluorenyl-9-methoxycarbonyl (Fmoc) protected amino acids (Bachem Ltd; St Helens, UK) (0.2 mmoles) were coupled to the amine functionalised glass surfaces in the presence of 0.4 mmoles 1-hydroxybenzotriazole (HOBt) and 0.4 mmoles N,N′-diisopropylcarbodiimide (DIC) in 10 ml N,N-dimethylformamide (DMF). Fmoc-amino acid coupling was carried out twice, by immersion in solution for 2½ hours in the first instance followed by rinsing with DMF, methanol, ethanol and DMF again, then immersion in fresh solution for approximately 16 hours, followed by rinsing as described above, producing Fmoc-amino acid-PEGA surfaces. Fmoc protecting groups were removed by immersion in 10% piperidine in DMF for 45 minutes and subsequent coupling of amino acids produced the desired surface bound peptide. Finally side-protecting groups were removed by immersion in aqueous trifluoroacetic acid (TFA, 50%) for 30 minutes. The aforementioned rinsing scheme was employed after piperidine treatment and DMF, methanol, ethanol and water used following TFA treatment.

### Enzyme Treatment of Surfaces

Three proteolytic enzymes, chymotrypsin, thermolysin and proteinase K, that are known to cleave peptide bonds involving F amino acids in the P1 position were tested for their ability to selectively hydrolyse Fmoc-F↓RGD bond¹⁵. All three enzymes have molecular masses of <38 kDa meaning PEGA is readily accessible to the enzymes^{4b}. Surfaces were incubated at 37° C. for 2 hours in enzyme solution (concentrations and activities are shown in Table 1) in phosphate buffer and the cleaved products were analysed by HPLC.

### HPLC Analysis

Surfaces were rinsed in a known volume (at least 4 ml) of a 50:50 mixture of acetonitrile (ACN) and water. The rinsing solution was kept and added to the enzyme solution that the surface was in. 1 ml of this solution was placed into an HPLC autosampler vial. The HPLC machine (manufactured by Dionex; California, USA) consisted of a P.680 HPLC pump joined to an ASI-100 Automated Sample Injector with a Dionex UVD17OU detector. The column was an EC 250/4.6 Nucleosil 100-5 C18, with an internal diameter of 4.6 mm and a column length of 250 mm containing particles of 5 μm in diameter. 100 μl was injected into the HPLC column at the start of each run and a buffer gradient run through the column, buffer A was 99.9% water and 0.1% TFA, buffer B was 99.9% ACN and 0.1% TFA. For quantification, three samples of each surface were produced and repeated three times.

### Dansyl Chloride Labelling

The homogeneity of the PEGA surfaces was characterized using dansyl chloride labelling of primary amines and analysis by fluorescence microscopy. Surfaces were rinsed in ethanol and DMF and then immersed in 2 ml of dansyl chloride solution: 10 ml DMF, 180 mg of dansyl chloride, 125 μl N′N-diisopropylethylamine (DIPEA) and left in the dark for 45 minutes. Surfaces were then rinsed in DMF, ethanol and water and viewed surfaces by fluorescence microscope (Eclipse 50i, Nikon; Melville, USA) and Lucia software.

### Interferometry

PEGA coatings were examined using an interferometer (MicroXam Interferometer, Phase Shift; Tucson, Ariz., USA) with ×50 magnification objective, a measurement area of 165×125 □m and a spatial sampling of 0.22×0.26 mm. Approximately half of the PEGA coating was removed using a scalpel and the glass/polymer interface was examined to determine the thickness of the polymer layer.

### Protein Adsorption Assay

Surfaces were incubated in 2 ml cell culture medium containing 10% foetal bovine serum at 37° C., 5% CO₂ and 95% humidity for 24 hours. The medium was then removed and the samples were rinsed in 1 ml of distilled water on a shaker for 15 minutes. Adsorbed protein was then desorbed by treatment with 6 M urea on a shaker for 30 minutes at room temperature. The urea protein solution was quantified using the Quant-iT™ Protein Assay Kit (Molecular Probes, Inc.; Paisley, UK) using the manufacturers protocol. All reagents were equilibrated at room temperature prior to use. The Quant-iT Protein Reagent was diluted 1:200 in the Quant-iT Protein Buffer, 190 μl of the diluted reagent was loaded into a 96-well plate, to which a further 10 μl of desorbed protein/urea sample solution was added. Fluorescence was measured using a fluorescence plate reader at 495/585 nm. Sample protein concentrations were determined using a standard curve of known protein (FBS)/urea concentrations. Three samples of each surface were produced and repeated three times.

### Cell Culture and Fluorescence Staining

Primary derived human osteoblasts (HOB) from femoral head trabecular bone were maintained in culture up to passage number 25 in Dulbecco's Modified Eagle Medium (DMEM, +1000 mg/L glucose, +GlutaMAX I, +pyruvate) supplemented with 10% (v/v) foetal bovine serum, 1% (v/v) antibiotic/antimycotic and 50 μg/ml ascorbic acid, at 37° C. and 5% CO₂. Near confluent flasks of HOB cells were rinsed with phosphate buffered saline (PBS) and incubated with 0.25% trypsin EDTA for 5 minutes, then resuspended in culture medium or serum-free culture medium. HOB cells were seeded onto all surfaces at a density of 100,000 cells/cm² and maintained in culture. For each experiment surfaces were prepared and experiments carried out in triplicate. Cell number and morphology were determined at 3 hours and 24 hours using a Leica® Inverted Microscope with digital camera and Spot Advanced software (version 3.2.1 for Windows, Diagnostic Instruments Inc.). Cells were counted in 5 random fields of view on each sample and the mean number taken. Cell number analysis was carried out using ImageTool Software (version 3.00, The University of Texas Health Science Centre in San Antonia, UTHSCSA). Spreading cells were distinguished by their polygonal morphology. One standard deviation was used as a measure of spread from the mean (n=15).

Cell morphology was examined by fluorescence staining as follows: Culture medium was removed and samples were rinsed twice with PBS. Formaldehyde (3.7%) was used to fix the cells, followed by further rinsing with PBS. The cells were permeabilised for 5 minutes using 0.1% Triton X100, followed by three further PBS rinses. Samples were then immersed for 30 minutes in PBS containing 1% bovine serum albumin (BSA). This solution was removed, and the actin filaments were then stained with fluorescein isothiocyanate (FITC)-conjugated phalloidin (10 μg/ml) (Invitrogen; Paisley UK) in PBS for 20 minutes at 4° C. Samples were mounted under glass coverslips with a drop of Prolong Gold antifade reagent containing DAPI (4,6-diamidino-2-phenylindole.2HCl, 10 μg/ml) (Invitrogen; Paisley, UK) to stain the cell nuclei. Cell morphology was then examined using fluorescence microscopy (Eclipse 50i, Nikon; Melville, USA) and Lucia software (version 4.82; Laboratory Imaging Ltd.).

### Results and Discussion

### Surface Characterisation

The thickness of the PEGA layers was determined using interferometry. The PEGA overlayer was removed on half of the sample using a scalpel and the interface examined. The distance between the PEGA overlayer and the glass substrate was determined to be the thickness of the PEGA coating, and was approximately 7 microns. When labelled with dansyl chloride, unmodified PEGA surfaces showed a homogenous distribution of chemically reactive primary amines at the micron scale.

Stepwise solid phase peptide synthesis was used to couple Fmoc-protected amino acids one-by-one to build up the desired peptide chains. The activity and specificity of the enzymes was determined by HPLC and is shown in Table 1. Proteinase K showed the highest cleavage but poor selectivity (cleaving both Fmoc-F↓RGD and Fmoc-FR↓GD) (entry 1), thermolysin also showed poor selectivity (entry 3) with chymotrypsin performing best (entry 2); however, some hydrolysis of Fmoc-FR↓GD was still observed.

This observation probably relates to traces of trypsin present in the chymotrypsin preparation. Indeed, trypsin-free chymotrypsin (TF-Ch) demonstrated higher selectivity for Fmoc-F and was used in further experiments (entry 4). A control experiment with Fmoc-^{D}FRGD-PEGA showed little hydrolysis (entry 5). The maximum amount of Fmoc-peptide that was cleaved from the surface was 4.08 nmol (proteinase K, determined by HPLC). This figure represents more than ½ the maximum loading of the polymer (calculated using a value of 0.23 mmol/g as the maximum loading of ˜0.03 g of PEGA (Mw=1900).

Table 1: Enzymatic hydrolysis of PEGA surface tethered peptides as analysed by HPLC. The maximum amount of Fmoc-peptide hydrolysed from the surface was 4.08 nmol (100%). The error corresponds to the standard deviation (n=9).

TABLE 1
Enzymatic hydrolysis of PEGA surface tethered
peptides as analysed by HPLC.

| | Enzyme, Concentration | Mw/ | Fmoc-F-OH | Fmoc-FR-OH |
|---|---|---|---|---|
| Entry | (units/mg) | kDa | (%) | (%) |
| 1 | Proteinase K, 30 | 27 | 60 ± 3.0 | 40 ± 8.0 |
| 2 | Chymotrypsin, 40 | 25 | 42 ± 1.8 | 7.5 ± 1.4 |
| 3 | Thermolysin, 40 | 37.5 | 40 ± 2.4 | 27 ± 3.5 |
| 4 | TF-Ch, 40 | 25 | 41 ± 3.2 | 3 ± 0.5 |
| 5† | TF-Ch, 40 | 25 | 3.5 ± 0.8 | 0.5 ± 0.1 |

The maximum amount of Fmoc-peptide hydrolysed from the surface was 4.08 nmol (100%). The error corresponds to the standard deviation (n = 9).
†The D form of phenylalanine was used

### Osteoblast Response to Enzyme-Responsive Surfaces

The cellular response to modified PEGA surfaces was studied using primary derived human osteoblasts. Osteoblasts did not attach to unmodified PEGA surfaces (FIG. 2, B) due to the non-fouling properties of PEGA, as observed previously for fibroblasts⁶. Introduction of RGD to PEGA promoted cell spreading to 40% (±5.5%) after 48 hours (FIG. 2, B). Fmoc-FRGD-PEGA showed little osteoblast spreading, demonstrating that the presence of Fmoc-F effectively inactivates the RGD functional peptide.

After exposure to TF-Ch, (Fmoc-F↓RGD, FIG. 2, B) osteoblast spreading increased to approximately 50% (±5%), which is not significantly different to the RGD-PEGA control surface (p>0.05 determined by the two-tailed student's t-test), while no cell attachment was observed when the non-enzyme cleavable Fmoc-^{D}FRGD sequence was employed. A control experiment consisting of an enzyme cleavable sequence with non-adhesive sequence (Fmoc-F↓RGE) showed little cell spreading after 48 hours. Similar figures for percentage cell spreading were seen after 5 days incubation. These data demonstrate that the surfaces were enzymatically switched by TF-Ch from inactive Fmoc-FRGD-PEGA to active RGD-PEGA surfaces.

The amount of adsorbed protein on PEGA surfaces was determined using the Quant-iT™ Assay Kit and is shown in Table 2. There was no significant difference (determined by student's t-test) in amount of total protein adsorbed between unmodified PEGA, Fmoc-FRGD-PEGA and Fmoc-F↓RGD-PEGA surfaces further confirming that osteoblasts attach specifically to Fmoc-F↓RGD-PEGA surfaces rather than by unspecific interactions with an adsorbed protein layer.

Table 2: The average amount of protein absorbed on various PEGA surfaces after 24 hours. The errors correspond to the standard deviations where n=9.

TABLE 2
The average amount of protein adsorbed on
various PEGA surfaces after 24 hours.

| | Amount of Protein | |
|---|---|---|
| Surface | (μg/mm²) | Non Cell Adhesive? |
| Glass | 0.240 ± 0.015 | NO |
| PEGA | 0.0180 ± 0.0009 | YES |
| Fmoc-FRGD-PEGA | 0.0189 ± 0.001 | YES |
| Fmoc-F↓RGD-PEGA | 0.0190 ± 0.0018 | NO |

The errors correspond to the standard deviations where n = 9.

Whilst chymotrypsin was used to activate the surface, another enzyme, trypsin was used to switch off cell attachment. In this experiment Fmoc-FRGD-PEGA surfaces were switched with TF-Ch and incubated with osteoblasts for 48 hours at which point the percentage of spreading cells was approximately 60 percent. Trypsin was then added to the system and after 1 hour the percentage of spreading cells fell to less than 1 percent. Washing of the surface with PBS removed the unattached cells and confirmed that the cells were no longer attached to the surface. To determine the nature of this inactivation, osteoblasts were re-seeded onto the surfaces to determine whether RGD groups remained intact, or if the trypsin treatment cleaved R from RGD (as predicted by the specificity of trypsin), thus inactivating the surface. The percentage of spreading cells on these surfaces was ˜35% (compared to ˜65% before trypsin treatment). These data indicate that cell detachment is mostly caused by the action of trypsin on cell focal adhesions but partly due to cleavage of R↓GD.

The light micrographs in FIG. 2, C show osteoblasts on Fmoc-FRGD-PEGA surfaces switched in situ by trypsin-free chymotrypsin after (i) 6 hours (ii) 24 hours (iii), 3 days and (iv) 5 days. The scale bars represent 50 μm. In the course of time, discrete areas of cell attachment appeared after 1 day, with further increases after 3 and 5 days while control experiments showed no significant cell spreading.

### (B) ENZYME-RESPONSIVE CELL ATTACHMENT TO PEG MONOLAYERS

### Materials and Methods

All chemicals and reagents, unless stated otherwise, were purchased from Sigma Aldrich Company Ltd. (Gillingham, UK) and used as received. All cell culture reagents, media and buffers were purchased from Invitrogen Ltd (Paisley, UK).

### Preparation of Surfaces

Borosilicate glass coverslips (Chance Glass Ltd; Malvern, UK. 12 mm diameter, No. 2 thickness) and all other glassware used were cleaned prior to use by immersion in Piranha solution, a 3:7 mixture of 30% hydrogen peroxide solution and concentrated sulphuric acid, for 30 minutes, followed by rinsing in copious amounts of deionised water, and drying in an oven at 100° C. overnight.

### Silanation and PEG Coupling

PEG monolayers were produced with reference to Piehler et al.¹⁹. Glass coverslips were modified with (3-glycidyloxypropyl) trimethoxysilane (GOPTS) by incubation in 100%

GOPTS at 37° C. for 1 hour to produce epoxy coated glass (FIG. 3, i). The coverslips were then washed in dry acetone and dried in a nitrogen gas flow. Surfaces were immediately treated with pure PEG diamine (n=18) by melting a layer of pure PEG powder on the surface at 75° C. for 48 hours (FIG. 3, ii). After which the surfaces were thoroughly washed in distilled water and dried under atmospheric conditions.

### Modification of PEG Surfaces

Solid phase peptide synthesis was used to couple amino acids or peptides to the terminal amine groups on PEG (FIG. 3, iii).

Fluorenyl-9-methoxycarbonyl (Fmoc)-peptides were produced either by stepwise solid phase synthesis or by a one step coupling of a preformed peptide via the terminal amine groups on PEG surfaces.

0.2 mmoles Fmoc protected amino acids or peptides (Bachem Ltd; St Helens, UK) were coupled to the amine-rich PEG surfaces in the presence of 0.4 mmoles 1-hydroxybenzotriazole (HOBt) and 0.4 mmoles N,N′-diisopropylcarbodiimide (DIC) in 10 ml N,N-dimethylformamide (DMF). All samples were rinsed with DMF, ethanol, methanol and DMF again. Fmoc-amino acid or peptide coupling was carried out twice, by immersion in solution for 2 hours in the first instance followed by rinsing as described above, then immersion in fresh solution for at least 16 hours, followed by rinsing as described above, producing Fmoc-amino acid or Fmoc-peptide surfaces. For stepwise solid phase peptide synthesis Fmoc protecting groups were removed by immersion in 10% piperidine in DMF for 30 minutes (FIG. 3, iv) and other side-protecting groups (O-t-Butyl (OtBu) on Aspartic acid D and Glutamic acid E; pentamethyl-dihydrobenzofuran-5-sulfonyl (Pbf) on Arginine, R; and t-Butyloxycarbonyl (Boc) on Tryptophan W) were removed by immersion in aqueous trifluoroacetic acid (TFA) (90%) for 30 minutes. The aforementioned rinsing scheme was employed following both protecting group removal stages. The structure of Fmoc-FRGD-PEG-epoxy silane is given in the bottom image of FIG. 3.

### Enzyme Treatment

Surfaces were incubated at 37° C. for 2 hours in 2 ml trypsin-free chymotrypsin (activity of 40 units/mg, 1 mg/ml) or serine elastase (15 units/mg, 2.5 mg/ml) enzyme solution and washed in distilled water and ethanol.

### Surface Analysis

### Protein Adsorption Assay

Surfaces were incubated in 2 ml cell culture medium containing 10% foetal bovine serum at 37° C., 5% CO₂ and 95% humidity for 24 hours. The medium was then removed and the samples were rinsed in 1 ml of distilled water on a shaker for 15 minutes. Adsorbed protein was then desorbed by treatment with 6M urea on a shaker for 30 minutes at room temperature. The urea protein solution was quantified using the Quant-iT™ Protein Assay Kit (Molecular Probes; Inc., Paisley, UK) using the manufacturers protocol. All reagents were equilibrated at room temperature prior to use. The Quant-iT™ protein reagent was diluted 1:200 in the Quant-iT Protein Buffer, 190 μl of the diluted reagent was loaded into a 96-well plate, to which a further 10 μl of desorbed protein/urea sample solution was added. Fluorescence was measured using a fluorescence plate reader at 495/585 nm. Sample protein concentrations were determined using a standard curve of known protein (FBS)/urea concentrations. Samples were prepared in triplicate and repeated three times.

### ToF SIMs Analysis

Secondary ion mass spectrometric analysis was carried out using a SIMS IV time-of-flight (ToF-SIMS) instrument (ION-TOF GmbH.; Munster, Germany) equipped with a gallium liquid metal ion gun and a single-stage reflectron analyser. Typical operating conditions utilised a primary ion energy of 15 kV, a pulsed target current of approximately 1.3 pA and post-acceleration of 10 kV. Low energy electrons (20 eV) were used to compensate surface charging caused by the positive primary ion beam on insulating surfaces. Large scale images were acquired by rastering the stage under the pulsed primary ion beam, using a raster of 0.5 mm². All doses were kept well below the static limit, with a maximum dose of 10¹² ions per cm² for both polarities combined. Acquisition of full raw datasets allowed for the retrospective construction of spectra from the imaged areas. Positive spectra were normalised to the intensity of the common C₂H₃⁺ fragment for comparison between samples.

### XPS Analysis

XPS was carried out on a Kratos Axis Ultra (Kratos Analytical Ltd; Manchester, UK) using a monochromated aluminium source, run at 150 W. A take-off angle of 90° was used, and all samples were analysed with charge neutralising electrons. The survey spectra were collected using a pass energy of 80 eV and the C1s core level spectra were collected at a pass energy of 20 eV. The spectra were charge corrected to position the C—C within the C1s core level at a binding energy of 285.0 eV. Symmetrical sum Gaussian/Lorenzian 30% peak shapes were used for all components and shifts presented relative to the C—C component at 285.0 eV.

### HPLC Analysis

Fmoc deprotection and enzyme efficiency was analysed by HPLC. Surfaces were rinsed in a known volume (at least 4 ml) of a 50:50 mixture of acetonitrile (ACN) and water. The rinsing solution was kept and added to the Fmoc-piperidine or enzyme solution that the surface was in and 1 ml of this solution was placed into an HPLC autosampler vial. The HPLC machine (manufactured by Dionex; California, USA) consisted of a P.680 HPLC pump joined to an ASI-100 Automated Sample Injector with a Dionex UVD17OU detector. The column was an EC 250/4.6 Nucleosil 100-5 C18, with an internal diameter of 4.6 mm and a column length of 250 mm containing particles of 5 μm in diameter. 100 μl was injected into the HPLC column at the start of each run and a buffer gradient run through the column, buffer A was 99.9% water and 0.1% TFA, buffer B was 99.9% ACN and 0.1% TFA. Molecules were identified by comparison with know standards and quantified using calibration curves. For quantification, samples were made in triplicate and repeated three times.

### Cell Culture

Primary derived human osteoblasts (HOB_{S}) from femoral head trabecular bone were maintained in culture up to passage number 25 in Dulbecco's Modified Eagle Medium (DMEM, +1000 mg/L glucose, +GlutaMAX I, +pyruvate) supplemented with 10% (v/v) foetal bovine serum, 1% (v/v) antibiotic/antimycotic and 50 μg/ml ascorbic acid, at 37° C. and 5% CO₂. Near confluent flasks of HOB cells were rinsed with phosphate buffered saline (PBS) and incubated with 0.25% trypsin EDTA for 5 minutes, then resuspended in culture medium or serum-free culture medium. HOB cells were seeded onto all surfaces at a density of 100,000 cells/cm² and maintained in culture. For each experiment surfaces were prepared and experiments carried out in triplicate. Cell number and morphology were determined using a Leica® Inverted Microscope with digital camera and Spot Advanced software (version 3.2.1 for Windows; Diagnostic Instruments Inc.). Cells were counted in 5 random fields of view on each sample and the mean number taken. Cell number analysis was carried out using ImageTool software (version 3.00,

The University of Texas Health Science Centre in San Antonia). Spreading cells were distinguished by their polygonal morphology. One standard deviation was used as a measure of spread from the mean (n=15).

### Results and Discussion

### Surface Analysis

### XPS Analysis of Single Amino Acid-PEG Surfaces

XPS analysis of piranha cleaned glass surfaces showed a small amount of carbon (˜12 at %) and nitrogen (˜2 at %) a large amount of oxygen (˜68 at %) and silicon (˜19 at %) as shown in FIG. 4. The silanation process increased the amount of surface carbon to ˜19 at % due to the carbon in the GOPTS molecule. The oxygen and silicon concentrations slightly decreased as the glass substrate signal was attenuated by this overlayer. The amount of nitrogen at the surface was roughly the same as that for the glass surfaces. The source of nitrogen in the glass is thought to be nitrates in the glass given the predicted removal of all organic nitrogen species by the Piranha cleaning procedure. After PEG coupling, the concentration of carbon and nitrogen species increased because of the high proportion of these elements associated with the PEG molecules. The PEG surfaces modified with an Fmoc-amino acid (Fmoc-Trp, FIG. 5) had an increased concentration of surface carbon, as expected, due to the presence of carbon in the Fmoc protecting group. The nitrogen was increased relative to the PEG surface, interpreted to represent the nitrogen from the amide bonds of the amino acid (Trp). The concentration of oxygen and silicon was lower on Fmoc-Trp surfaces than PEG surfaces, interpreted as masking of the glass and PEG by the Fmoc and Trp groups. The concentration of nitrogen was higher at the Fmoc-Trp surface than the previous stages in surface production, due to the nitrogen content of the Trp. Following Piperidine treatment (Trp surface, FIG. 5) the concentration of carbon was higher than for Fmoc-Trp surfaces. This may relate to a pick up of hydrocarbon contamination.

Peak fitting of the C1s peaks revealed the functional nature of the carbon species for each surface (FIG. 6). Glass had a low amount of C—C bonds (˜6 at %), C—O and NC═O groups (˜3 at % and ˜0.7 at % respectively) and no carbamate groups (O—C(═O)—N), which link the fluorenyl group to the peptide, and are associated with the presence of Fmoc groups²⁰. After GOPTS and PEG coupling the amount of all carbon containing species increased with the exception of carbamate, which had a negligible amount on GOPTS. Fmoc-Trp samples had a large amount of C—C and C—O groups, a moderate amount of NC═O and a relatively high amount of carbamate groups, showing the successful coupling of Fmoc-Trp molecules. After Fmoc deprotection the carbamate group was not completely removed, but was reduced (significantly different, as determined by the student's t-test). An increase in NC═O groups associated with the coupled amino acid was seen for Trp samples.

### ToF SIMS Analysis

ToF SIMS analysis showed positive ion intensity maps for each of the surfaces at selected masses. The peak at m/z=28 represents silicon and is most intense on glass surfaces as expected, m/z=45 represents C₂H₅O and is indicative of PEG groups (C₂H₅O⁺). The peak at m/z=130 represents tryptophan²¹ and was only present at significant intensities for Fmoc-Trp and Trp surfaces. The m/z=179 ion fragments are associated with the Fmoc group (C₁₄H₁₁⁺)²². The m/z=179 intensity maps follow the pattern expected from the molecular structure shown in FIG. 3; low (background) levels on glass, GOPTS and PEG (surfaces that have not had Fmoc exposure), a high intensity on the Fmoc-Trp surface, confirming that Fmoc-Trp was successfully attached to the PEG surface, and a low intensity on Trp surfaces, indicating the Fmoc decoupling step was efficient.

After the coupling of a single amino acid was confirmed, we next looked at ToF SIMS spectra for PEG surfaces modified in one step with the full peptide sequence Fmoc-FRGD. FIG. 6 shows ToF SIMS spectra for Fmoc-FRGD-PEG+chymotrypsin treatment (A), Fmoc-FRGD-PEG with no enzyme treatment (B) and Fmoc-Trp as a comparison (C).

Fmoc fragments occur at 179 m/z (large peak in FIG. 6, C). It is clear that the single step coupling of Fmoc-FRGD to PEG was not successful to any great degree in the areas analysed as there are no significant peaks at 179 m/z in A or B.

### HPLC Analysis

HPLC was used determine the efficiency of five coupling steps of amino acids to PEG monolayers. FIG. 7 shows that for each coupling step a similar amount of Fmoc was removed from the surface. Assuming the Fmoc deprotection step was close to 100% efficient, the efficiency of peptide formation is likely to be similarly high.

HPLC was also used to determine the efficiency of enzyme reactions. For the Fmoc-FRGD-PEG+chymotrypsin system, cleavage of Fmoc-F from the surface was 0.42±0.009 nmol. This figure corresponds to approximately 60% of the total loading of the surface (assuming average values in FIG. 8 correspond to 100% efficiency of Fmoc deprotection). For the Fmoc-AARGD-PEG+elastase system, cleavage of Fmoc-A from the surface was 0.64±0.004 nmol corresponding to approximately 92% of the total loading of the surface (again assuming 100% efficiency of Fmoc deprotection).

### Protein Adsorption

The Quant-iT™ protein assay kit was used to determine the amount of adsorbed protein on various surfaces as shown in Table 3.

Table 3: The average amount of protein absorbed to various surfaces after 24 hours. The errors correspond to the standard deviations where n=9.

TABLE 3
The average amount of protein adsorbed
to various surfaces after 24 hours.

| | Amount of Protein | |
|---|---|---|
| Surface | (μg/mm²) | Non Cell Adhesive? |
| Glass | 0.240 ± 0.015 | NO |
| PEG₁₈ | 0.0126 ± 0.001 | YES |
| Fmoc-Trp-PEG₁₈ | 0.0156 ± 0.001 | YES |
| Trp-PEG₁₈ | 0.0166 ± 0.0008 | YES |

The errors correspond to the standard deviations where n = 9.

The amount of protein adsorbed on glass surfaces was higher than for all PEG-based surfaces. The higher amount of adsorbed protein on glass than PEG is reflected by the fouling property of glass. The amount of adsorbed protein is similar for the PEG-based surfaces, although the presence of Fmoc-Trp or Trp increases the amount of adsorbed protein but does not affect the non-fouling property of the surface. It is likely that the presence of Fmoc-Trp or Trp reduces the interactions between surface bound PEG chains, allowing more proteins to adsorb. However this increase in protein adsorption was not enough to allow cells to attach. The amount of protein at the surface is similar to values quoted by Benesch et al.²³ (˜800 ng/cm² for serum adsorption on PEG monolayers).

### Cell Culture

### Fmoc-FRGD-PEG+Chymotrypsin System. 1. Preformed Peptide

Osteoblasts seeded onto PEG₁₈-modified glass were not spread after 24 hours. Cells could be washed from the surfaces using PBS with minimum effort indicating there was no significant cell attachment to PEG surfaces. PEG₁₈ coverage was sufficient to resist cell spreading for up to 5 days (longest time period studied). After 24 hours the percentage of spreading osteoblasts at the surface of PEG-modified glass was approximately 3% (FIG. 8). Although PEG surfaces when modified with the preformed peptide Fmoc-FRGD provoked significantly more cell spreading than unmodified PEG surfaces, the value (˜9%) is less than Fmoc-F↓RGD-PEG surfaces, indicating that Fmoc-F inactivated RGD groups. After treatment with chymotrypsin (Fmoc-F↓RGD-PEG surfaces), the percentage of spreading cells rose to approximately 60% indicating there was sufficient number of RGD groups at the surface to induce cell spreading. Osteoblasts on Fmoc-F↓RGD-PEG surfaces could not be removed by light washing in PBS showing that cells were attached with reasonable strength.

Cell culture results have shown that Fmoc-F↓RGD-PEG surfaces induce osteoblast spreading compared to Fmoc-FRGD-PEG and PEG controls. This cell spreading was localised in discrete areas, thus it is likely that, contrary to ToF SIMS data, the coupling of the whole Fmoc-FRGD molecule was successful, albeit in small discrete areas. Hence the one-step coupling of Fmoc-FRGD was inefficient, probably due to its large size and also possibly due to inaccessibility of the terminal amine groups on PEG. Stepwise coupling of Fmoc-amino acids has been shown by XPS, ToF SIMs and HPLC to be effective for the formation of peptides on PEG surfaces. It would appear that the size of Fmoc-amino acids is sufficiently small so that they can be successfully coupled to surface bound PEG.

It is clear from HPLC data and cell culture experiments that the chymotrypsin treatment effectively removed Fmoc-F from the surface of Fmoc-FRGD-PEG samples. In other words the surface was switched from bioinert to bioactive by chymotrypsin treatment. However the coupling of a large Fmoc-peptide was inefficient and thus stepwise attachment of Fmoc-amino acids would seem to be preferable for PEG monolayer systems.

### Fmoc-FRGD-PEG+Chymotrypsin System. 2. Stepwise Peptide Synthesis

To increase the amount of Fmoc-FRGD groups at the surface, stepwise peptide synthesis was used to create the desired peptide. When osteoblasts were seeded onto the stepwise-formed Fmoc-F↓RGD-PEG surfaces (85% ±4.2) the percentage of spreading cells was greater than that for the preformed Fmoc-F↓RGD-PEG peptide (62% ±7). This result can be explained by the different sizes of the coupling molecules. Fmoc-amino acids are smaller, meaning that they are more likely to be correctly orientated to react with the surface than the larger Fmoc-FRGD molecule. Hence the overall coverage of Fmoc-FRGD was greater on stepwise formed surfaces than surfaces made using preformed Fmoc-FRGD. Chymotrypsin is a model enzyme only and is not associated with any major diseases. As such, a system was developed using a more disease specific enzyme (serine elastase) and using stepwise solid phase synthesis to efficiently attach amino acids one-by-one.

### Fmoc-AARGD-PEG+Elastase System

PEG surfaces were resistant to cell spreading up to 5 days (FIG. 9). Fmoc-AARGD-PEG surfaces with no enzyme treatment did not induce cell spreading to a large degree at any time points, although statistically more than PEG surfaces (determined by two-tailed student's t-test, p<0.05). It is likely that the introduction of Fmoc-AARGD to PEG surfaces affected the resistance of PEG to cell attachment by reducing the interactions between PEG chains and thus slightly reducing the repulsion of proteins/cells. The Fmoc-A↓ARGD-PEG surfaces (with elastase treatment) promoted a large percentage of spreading cells compared to Fmoc-AARGD-PEG and PEG surfaces. The cell response, together with HPLC data, confirms that the surfaces had been successfully switched from bioinert to bioactive. ARGD-PEG positive control surfaces showed statistically similar cell responses to enzyme treated surfaces, showing that the presence of alanine (A) at the carboxyl end of RGD does not appear to adversely affect its activity (˜95% spreading cells after 24 hours). Light micrographs of cells on various PEG surfaces are shown in FIG. 10. Osteoblasts remained rounded on PEG surfaces at all time points. Similarly the majority of cells on Fmoc-AARGD-PEG were rounded at the time points examined and could be removed by gentle washing in PBS. ARGD-PEG control surfaces induced cells to attach and spread (unable to remove by gentle PBS washing) showing a polygonal morphology. Cells on Fmoc-A↓ARGD-PEG had a similar morphology to cells on the ARGD-PEG control surfaces.

The percentage of spreading cells for the Fmoc-AARGD-PEG+elastase system was higher than for the Fmoc-FRGD-PEG+chymotrypsin system. This may have been due to the efficiency of the enzymes; elastase cleaved Fmoc-A↓ARGD more readily than chymotrypsin cleaved Fmoc-F↓RGD. It also seems likely that the inefficiency of attachment of Fmoc-FRGD in one step, as confirmed by ToF SIMS, led to fewer RGD groups at the surface than for the step-wise attachment of amino acids.

### REFERENCE

(1) 1(a) Lahann, J.; Langer, R. MRS Bull. 2005, 30, 185-188.; (b) Mrksich, M. MRS Bull. 2005, 30, 180-184.; (c) Yang, J.; Yamato, M.; Okano, T. MRS Bull. 2005, 30, 189-193.; (d) Nakanishi, J.; Kikuchi, Y.; Takarada, T.; Nakayama, H.; Yamaguchi, K.; Maeda, M. J. Am. Chem. Soc. 2004, 126, 16314-16315.; (e) Kim, M. R.; Jeong, J. H.; Park, T. G. Biotechnol. Prog. 2002, 18, 495-500.
(2)(a) Ulijn, R. V. I J. Mat. Chem. 2006, 16, 2217-2225.; (b) Wu, L.-Q.; Payne, G. F. Trends Biotechnol. 2004, 22, 593-599.; (c) Hubbell, J. A. Curr. Opin. Biotech. 1999, 10, 123-129.; (d) Langer, R.; D. A. Tirell. Nature, 2004, 428, 487-492.; (e) Lutolf, M. P.; Hubbell, J. A. Nature Biotech. 2005, 23, 47-55.; (f) Harris, T. J.; von Maltzahn, G.; Derfus, A. M.; Ruoslahti, E.; Bhatia, S. N. Angew. Chem., Int. Ed. 2006, 45, 3161-3165.
(3) Dalsin, J. L and Messersmith P. B. Materials Today. September 2005, 38-46.
(4)(a) Meldal, M. Tetrahedron Lett. 1992, 33, 3077-3080.; (b) Halling, P. J., Ulijn, R. V., Flitsch, S. L., Curr. Opin. Biotechnol., 2005, 16, 1-8.
(5) Zourob, M.; Gough, J. E.; Ulijn, R. V.; Adv. Mater. 2006, 18, 655-659.
(6) Yamada, K. M. J. Biol. Chem.1991, 266 (2), 12809-12812.
(7) Singer, et al., J. Cell Biol. 1987, 104, 573-584.
(8) Kleinmann H. K. Vitamins and Hormones. 1993, 47,161-186.
(9) Hubbell, J. A. Ann. N.Y. Acad. Sci. 1992, 665, 253-258.
(10) Koliakos, G. G. J. Biol. Chem. 1989, 264, 2313-2323.
(11) Elliot et al. European Cells and Materials. 2002, 4, suppl 2, 46.
(12) Tashiro et al. J. Biol. Chem. 1989, 264 (27), 16174-16182.
(13) Huang J,.K. and Jan C, R. Drug Development Research. 2000, 50 (2), 147-152.
(14) Merrifield, R. B.; J. Am. Chem. Soc. 1963, 85, 2149-2154.
(15)(a) Schechter, I.; Berger, A. Biochem. Biophys. Res. Commun., 1967, 27, 157-162.; (b) Matsubara, H.; Sasak, R.; Singer, A.; Jukes, T. H. Arch. Biochem. Biophys. 1965, 115, 324-331. (c) Kraus, E.; Femfert, U. Hoppe Seylers Z Physiol. Chem. 1976, 357, 937-947.
(16) Hersel, U.; Dahmen, C.; Kessler, H. Biomater. 2003, 24, 4385-4415.
(17) Maroudas N. H. Theor. Biol. 1975, 49, 417-424.
(18) Burch, R. M.; Weitzberg, M.; Blok, N.; Muhlhauser, R.; Martin, D.; Farmer, S. G.; Bator, J. M.; Connor, J. R.; Ko, C.; Kuhn, W.; McMillan, B. A.; Raynor, M.; Shearer, B. G.; Tiffany, C.; Wilkins, D. E. Proc. Nat. Acad. Sci. USA, 1991, 88, 355-359.
(19) Piehler J, Brecht A, Valiokas R, Liedberg B, Gauglitz G. Biosensors & Bioelectronics 2000, 15: 473-481
(20) Rawsterne R E, Todd S J, Gough J E, Rutten F J M, Alexander M R, Ulijn R V. Acta Biomaterialia 2007, 3: 715-721.
(21) Wagner M S, Castner D G. Langmuir, 2001. 17: p. 4649-4660
(22) Rawsterne R E, Gough J E, Rutten F J M, Pham N T, Poon W C K, Flitsch S L, Maltman B, Alexander M R, Ulijn R V. Surface and Interface Analysis 2006, 38: 1505-1511
(23) Benesch J, Svedhem S, Svensson S C T, Valiokas R, Liedberg B, Tengvall P. Journal of Biomaterials Science, Polymer Edition 2001, 12: 581-597

1.-63. (canceled)

64. A substrate having a biomolecule immobilized thereon, wherein the biomolecule is connected via an enzyme cleavable link to a blocking moiety such that cleavage of the link causes removal of the blocking moiety.

65. The substrate according to claim 64, wherein the substrate comprises a polymer selected from the group consisting of collagen, gelatin, hyaluronan, cellulose, chitin, dextran, fibrin, casein, and a synthetic polymer selected from the group consisting of polylactide (PLA), polyglycolide (PGA), poly(lactide-co-glycolide) (PLGA), poly(e-caprolactone), polydioxanone, polyanhydride, poly(ethylene terephthalate), poly(urethane), poly(methylmethacrylate), poly(styrene), trimethylene carbonate, poly(β-hydroxybutyrate), poly(g-ethyl glutamate), poly(DTH iminocarbonate), poly(bisphenol A iminocarbonate), poly(ortho ester), polycyanoacrylate, polyphosphazene, and poly(ethylene glycol)-acrylamide (PEGA); a ceramic; and a metal.

66. The substrate according to claim 64, wherein the biomolecule is covalently or non-covalently bound to the substrate.

67. The substrate according to claim 64, wherein an anti-fouling film is applied to at least part of the surface of the substrate onto which the biomolecule is immobilized, and wherein the anti-fouling film is selected from the group consisting of (i) an anti-fouling film comprising a hydrophilic polymer selected from group consisting of polyacrylate, phosphocholine, poly(ethylene) glycol (PEG), amino-functionalized PEG, 3,4-dihydroxy-L-phenylalanine (DOPA)-PEG and polyethylene glycol acrylamide (PEGA); and (ii) an anti-fouling film comprising an oligosaccharide or polysaccharide.

68. The substrate according to claim 64, wherein the biomolecule is selected from the group consisting of a lipid; phospholipid; glycolipid; sterol; vitamin; hormone; neurotransmitter; carbohydrate; monosaccharide; disaccharide; phosphate; amino acid; nucleic acid; nucleotide; peptide, wherein the peptide (i) has less than twelve amino acid residues and/or (ii) comprises a cell attachment recognition motif selected from the group consisting of a fibronectin motif, laminin motif, and collagen motif, or is a peptide comprising an anti-inflammatory sequence; oligopeptide; polypeptide; and protein.

69. The substrate according to claim 64, wherein the enzyme cleavable link is located between the blocking group and the biomolecule.

70. The substrate according to claim 64, wherein the enzyme cleavable link is located within the blocking moiety, and wherein the enzyme cleavable link is optionally a peptide, ester, glycoside or oligonucleotide.

71. The substrate according to claim 64, wherein the enzyme cleavable link contains an enzyme recognition motif for an oxidoreductase; transferase; hydrolase, wherein the hydrolase is selected from the group consisting of an aspartic-, glutamic-, serine-, cysteine-, metallo- and threonine-protease; lyase; isomerise and ligase; and optionally an amino acid having an aromatic side chain located at P1 of the enzyme cleavable link.

72. The substrate according to claim 64, wherein the biomolecule is a peptide comprising arginine-glycine-aspartic acid (RGD) connected to an enzyme cleavable link comprising phenylalanine (F) or consisting of phenylalanine (F), such that an enzyme can selectively hydrolyze the arginine-phenylalanine bond.

73. The substrate according to claim 64, wherein the blocking moiety sterically inhibits the biomolecule.

74. The substrate according to claim 64, wherein the blocking moiety is bioactive following cleavage.

75. The substrate according to claim 64, wherein the substrate is an in vitro cell culture substrate selected from the group consisting of a cell/tissue culture flask, cell/tissue culture plate, cell/tissue culture dish, Petri dish, microcarrier, and macrocarrier.

76. The substrate according to claim 64, wherein the substrate is at least a part of a surface of a medical device, a biomaterial, or a prostheses.

77. A method of enhancing cell adhesion to a substrate, the method comprising the steps of:
i) immobilizing a biomolecule onto the substrate, the biomolecule comprising a cell recognition motif and being connected via an enzyme cleavable link to a blocking moiety such that cleavage of the link causes removal of the blocking moiety and subsequent activation of the biomolecule; and
ii) exposing the biomolecule to an enzyme capable of cleaving the link.

78. The method according to claim 77, wherein:
i) the biomolecule is a peptide, said peptide comprising arginine-glycine-aspartic acid (RGD), and/or wherein the peptide is linked to a blocking moiety comprising N-fluorenylmethoxycarbonyl (Fmoc); and/or
ii) the enzyme-cleavable link comprises phenylalanine (F), optionally located in the P1 position; and/or
iii) the enzyme is an exogenous or endogenous enzyme; and/or
iv) the substrate is an in vitro cell culture substrate or is at least a part of a surface of a medical device.

79. A method of attenuating an inflammatory response in a subject following implantation of a medical device, the method comprising the step of immobilizing a biomolecule onto a surface of the device, wherein the biomolecule is connected via an enzyme cleavable link to a blocking moiety where cleavage of the link causes removal of the blocking moiety and activation of the biomolecule to an activated biomolecule, and wherein the activated biomolecule is an anti-inflammatory agent.

80. The method according to claim 79, wherein:
i) the activated biomolecule comprises a lysine-proline-valine (KPV); and/or
ii) the cleavage further causes activation of the blocking moiety, wherein the blocking moiety optionally comprises fluorenylmethoxycarbonyl (Fmoc), and wherein the activated blocking moiety is an anti-inflammatory agent.
